SEQUENCE LISTING (1 sequence)

SEQ ID NO: 1 - DNA, 5314 bp, UNKNOWN
  160728 bac SXT6
  attB1 recombination site (1)..(25)
  CV-A16 P1 (107)..(2695)
  IRES (2696)..(3281)
  EV-A71 3C-3D (3282)..(5222)
  attB2 recombination site (5289)..(5314)
acaagtttgtacaaaaaagcaggctagatcttaatacgactcactatattaattaaggatcccaaaaaattgaaattttattttttttttttggaatataaataatatgggctcacaagtcagcacgcaacgcagcggaagccacgagaacagcaactcggcatcggaaggctctactatcaactacactacaatcaactactacaaggacgcgtacgctgccagtgctggtaggcaggacatgtcgcaagatcctaagaaattcaccgaccccgtgatggatgtcatccatgagatggctcctcccttgaagtccccaagcgccgaagcatgcggctactccgacagagtggcccagctgacaatcggaaacagcacgattaccactcaagaggcagcgaacatcgtgattgcttacggcgagtggccagaatactgtccggacacagatgcgacggctgtcgacaagcctacccgccctgatgtctccgttaacagattcttcaccctggacactaagtcctgggccaaggatagcaaaggatggtactggaaattccctgacgtgctcactgaagtgggtgtcttcggccagaacgctcaattccactacttgtaccgttctggcttctgcgttcatgtgcagtgtaacgcctcaaagttccaccaaggagcactgctcgtcgcggttctgccagagtacgtcctcggtaccatcgctggtggcactggcaacgaaaacagccatccaccgtacgcgacaacgcagcctggacaagttggtgctgtgctgactcacccctacgtcctcgacgccggtatcccattgtctcagctgacagtttgccctcatcaatggattaacctgaggacaaacaactgtgctacgatcattgtgccatacatgaacaccgtcccgttcgactctgccctgaaccactgcaacttcggattgctggtcatcccagtggtcccgctcgatttcaacgccggtgcaacttcagagatcccaattacagtgacgatcgccccgatgtgtgctgaattcgctggactgaggcaggcagtcaagcaaggaatccctaccgagttgaaacccggtactaaccagttcctgaccactgacgatggcgtgagcgctcctatcctccccggattccaccccacccctcccatccatattccaggagaggtccacaacctcttggaaatctgcagggttgagacaattttggaagtgaacaacctgaagacgaacgagacaacgcctatgcagagattgtgcttccccgtgagtgtccaatcgaaaacaggcgaactgtgcgctgccttccgcgctgacccaggccgtgatggaccttggcagagtaccatcctcggtcaattgtgtcgctactacacccagtggtctggctcactcgaagtgactttcatgttcgccggatccttcatggcaaccggcaagatgttgatcgcctacactccaccgggaggtaacgtgcctgctgaccgtatcaccgctatgctgggtactcacgttatttgggatttcggcttgcaatccagcgtgacactggttgtgccatggatctcaaacactcactacagggctcatgctagggcaggatacttcgactactacaccactggtatcattaccatatggtaccagacgaactacgtcgttcctatcggagctcccacaacggcctacattgtggcactggcagcggctcaagacaacttcaccatgaagctctgcaaagacacagaggatatcgaacagacggcgaacattcaaggtgaccccatcgctgacatgattgatcagacagtcaacaaccaagttaaccgctccctgactgctctccaggtgttgcctactgcagcaaacactgaggcctcttcacaccgtctcggcacaggagtggtccccgcattgcaagcggctgaaaccggagcgagttcgaacgcttccgacaagaacctgatcgagacccgctgcgtcctcaaccaccatagcacacaggaaacggccattggaaacttcttctctcgtgcaggtctggtttcaatcattactatgcctaccactggcacccagaacactgacggatacgtcaactgggacatcgatctcatgggctacgctcaattgcgccgtaagtgtgagctgttcacctacatgcgcttcgatgccgaattcactttcgttgtggcaaaacctaacggagagctggtgccccagctgctccaatacatgtacgtccctcccggtgctcctaagccaacttcccgtgacagcttcgcctggcagaccgcaactaaccctagtgttttcgtgaaaatgacagatccaccggcccaggtctcggttccattcatgtctccggcatcagcgtaccaatggttctacgacggttaccccaccttcggcgagcacctccaggctaacgacttggattacggccaatgcccaaacaacatgatgggaaccttctctatcaggacagtgggtacggaaaagagtccccattcgattactctgagggtctacatgagaatcaaacacgttagagcctggattccaaggccgctcagaaaccagccatacttgttcaagactaaccctaactacaaaggcaacgatattaagtgtacctcgactagccgtgataaaattactactctgtaagcccctctccctcccccccccctaacgttactggccgaagccgcttggaataaggccggtgtgtgtttgtctatatgtgattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtagtcaacaaggggctgaaggatgcccagaaggtaccccattgtatgggaatctgatctggggcctcggtgcacatgctttacatgtgtttagtcgaggttaaaaaagctctaggccccccgaaccacggggacgtggttttcctttgaaaaacacgatgataagcttgccacaaatgggtccaagtcttgactttgctctttccctgctgagaaggaacatcaggcaagtccaaacagatcagggccattttaccatgttgggtgtcagggatcgcttggctgttctcccaaggcacgcacagcccggcaagactatttgggtggagcacaaacttgtgaacgtcctcgacgcaatcgagctggtggatgaacagggcgttaatttggaactcacactggtgacacttgacactaatgaaaaatttagagatatcaccaagttcattccagagaccattagtggcgctagtgatgcaactttggtgatcaatacagaacatatgccatcaatgtttgtcccagtgggcgacgtcgtgcagtatggattcttgaaccttagtggaaagccaacacataggaccatgatgtacaatttccctacaaaagcaggacaatgtggaggtgtggtcacatccgtcggtaagatcgttggtattcacattggcggcaacggccgccaaggtttctgtgctggtttgaagaggagttacttcgcaagtgtgcagggtgagatccaatgggtgaaacctaacaaagaaactggtagactgaacatcaatggaccaactcgcactaagctggagcctagtgtgtttcatgaagtgtttgaaggcaataaggaaccagcagtcttgacaagtaaagaccctagactggaggtcgactttgagcaagccctgttttccaagtatgtgggcaatgttttgcatgagcccgatgaatacgtgactcaagctgccctccactatgcgaatcaactcaaacagttggacataaacactagcaagatgagcatggaggaagcgtgctatggcactgagaacctggaagcaattgatctctgtactagtgccggatatccatacagcgcccttggcatcaagaaaagagacattctcgaccccgtaaccagggatgtgtctaagatgaaattctatatggacaaatacggcctcgatctgccatactccacctacgtaaaggatgagcttcgctctctggataaaatcaagaaaggaaagtcacgcctgatagaggctagtagcttgaatgactctgtctacctcagaatgacttttggccacctttacgaggtatttcatgctaaccctggcactgtgaccggttccgcagtgggttgcaacccagacgtgttttggagtaaacttccgatccttctgcctggctcactctttgcctttgactactcaggatatgatgctagcctcagcccggtatggttcagggcccttgaaattgtgttgcgcgaaattggctatccagaagaggccgtgtcccttatagaaggaatcaaccacactcaccacgtgtaccgcaacaaaacatactgtgtacttggtggcatgccctcaggctgttctggtacttccatctttaactcaatgatcaacaacatcatcattagaaccctcttgatcaaaacctttaagggaatagacctggatgagttgaacatggtggcctatggcgacgatgtgttggctagttatccctttcctatagattgccttgagttggctaagactggcaaagagtatggtttgactatgacgcctgcagacaaatcaccctgtttcaatgaagtgacgtgggaaaatgctaccttccttaagagaggcttcttgccagaccaccaatttccattcttgattcaccctacgatgcccatgagagagatccatgagtccattcgctggactaaagacgcacgcaatacccaagaccacgtgcgctccctgtgtctgttggcatggcacaacggtaaggatgaatatgagaaatttgtgagtgcaattagatcagttccagttggaaaagcgttggccattcctaattttgagaatttgagaagaaattggctcgaattgttttaaatcgatttaattaatagcataaccccttggggcctctaaacgggtcttgaggggttttttggaattcacccagctttcttgtacaaagtggt

(19) United States
(12) Patent Application Publication
(10) Pub. No.: US 2019/0247488 A1
(43) Pub. Date: Aug. 15, 2019

(54) EXPRESSION CASSETTES AND METHODS FOR OBTAINING ENTEROVIRUS VIRUS-LIKE PARTICLES

(71) Applicant: SENTINEXT THERAPEUTICS Sdn Bhd, PENANG (MY)

(72) Inventor: Mary Jane CARDOSA, KUALA LUMPUR (MY)

(21) Appl. No.: 16/339,520

(22) PCT Filed: Sep. 21, 2017
(86) PCT No.: PCT/MY2017/050060

§ 371 Date: Apr. 4, 2019

(30) Foreign Application Priority Data
Oct. 7, 2016 (MY) PI 2016703712

(51) Int. Cl. A61K 39/125; A61P 31/14; C12N 15/85

(52) U.S. Cl. CPC A61K 39/125; C12N 15/85; A61P 31/14

(57) ABSTRACT

This invention relates to Virus-Like Particles (VLPs) derived from Enterovirus and vaccines comprising such VLPs which elicit immune responses and/or protective neutralizing antibody responses directed against an Enterovirus. The instant invention provides expression cassettes and methods for making Enterovirus CV-A16 VLPs and compositions enriched in CV-A16 VLPs which exhibit conformational epitopes which elicit immune responses and/or neutralizing antibody responses which may be used in vaccines directed against Enterovirus CV-A16.

### TECHNICAL FIELD OF THE INVENTION

This invention relates to Virus-Like Particles (VLPs) derived from Enterovirus and vaccines comprising such VLPs which elicit immune responses and/or protective neutralizing antibody responses directed against an Enterovirus.

### BACKGROUND OF THE INVENTION

Enterovirus is a genus of viruses belonging to Picornavirus, family Picornaviridae. Enterovirus represents a genus of a large and diverse group of small RNA viruses characterized by a single positive-strand genomic RNA. All enteroviruses contain a genome of approximately 7,500 bases and are known to have a high mutation rate due to low-fidelity replication and frequent recombination. After infection of the host cell, the genome is translated in a cap-independent manner into a single polyprotein, which is subsequently processed by virus-encoded proteases into the structural capsid proteins and the nonstructural proteins, which are mainly involved in the replication of the virus.

The enteroviruses are associated with several human and mammalian diseases. Enteroviruses are classified into 12 species as follows: Enterovirus A, Enterovirus B, Enterovirus C, Enterovirus D, Enterovirus E, Enterovirus F, Enterovirus G, Enterovirus H, Enterovirus J, Rhinovirus A, Rhinovirus B and Rhinovirus C.

Within the twelve species of Enterovirus there are many serotypes. Serologic studies have distinguished numerous Enterovirus serotypes on the basis of antibody neutralization tests.

Species Enterovirus A includes, for example, serotypes EV-A71 (also referred to as EV71 or HEV71), EV-A76, EV-A89, EV-A90, EV-A91, EV-A92, CV-A16 (Coxsackievirus A16), CV-A5, CV-A6, and CV-A10.

Species Enterovirus C exhibits 23 serotypes, which include, for example, PV-1 (Poliovirus 1), PV-2, PV-3, CV-A20, CV-A21, EV-C95, EV-C96, EV-C99, EV-C102, EV-C104, EV-C105, and EV-C109.

Serotypes EV-D68, EV-D70, and EV-D94 are classified under the species Enterovirus D.

All members of the genus Enterovirus, including EV-A71, polioviruses and Coxsackievirus A16 have a single stranded positive sense RNA genome, which has a single open reading frame encoding a polyprotein, P1, consisting of the capsid structural proteins VP0, VP3 and VP1, and several non-structural proteins including the viral proteases 3C and 3CD which are responsible for cleaving the polyprotein P1 into the individual capsid proteins VP1, VP3 and VP0, which VP0 is eventually cleaved into VP2 and VP4 after viral RNA encapsidation. The capsid proteins, VP0, VP1 and VP3, may assemble into virus-like particles (VLPs) without encapsidation of the genome, but cleavage of VP0 into VP2 and VP4 occurs after RNA encapsidation during maturation of the native virus. (CHUNG, et al., World J Gastroenterol 12(6):921-927, 2006)).

Diseases caused by enterovirus infection include poliomyelitis which is the most notable disease caused by an Enterovirus infection. Examples of other diseases are aseptic meningitis, hand, foot and mouth disease (HFMD), conjunctivitis, respiratory illnesses and myocarditis. Nonspecific febrile illness is, however, the most common presentation of an Enterovirus infection.

Infection with Enterovirus C and especially polioviruses have been a widespread problem, and epidemics of poliomyelitis have historically been a major global health problem causing millions of deaths during the 20^{th} century. Inactivated whole virus vaccines have been used for mass immunization and are currently available and used for prophylaxis against poliovirus infection. Good results, leading to eradication of poliomyelitis in most countries of the world, have been obtained with inactivated poliomyelitis vaccines, which may be prepared according to a method which has been developed by Jonas Edward Salk and has been improved later in several aspects. Generally, these vaccines contain a mixture of inactivated polio viruses of strains Mahoney, MEF1 and Saukett. Although attenuated poliovirus serotypes PV-1, PV-2 and PV-3 strains (Sabin) have been produced and used as an attenuated oral polio vaccine, the attenuated Sabin vaccine occasionally produces revertants leading to what has been referred to as Vaccine Associated Paralytic Polio (VAPP). Typically, vaccination with the individual polypeptides of polioviruses in the form of a subunit vaccine has shown that the isolated polypeptides are not capable of raising neutralizing antibodies in animals (MELOEN, et al., J. Gen. Virol. 45:761-763, 1979).

Otherwise, enteroviruses are the most common causes of aseptic meningitis in children. In the United States, enteroviruses are responsible for 30,000 to 50,000 cases of meningitis. Encephalitis is a rare manifestation of an enterovirus infection; but when it occurs, the most frequent enterovirus found to be causing the encephalitis is echovirus 9. Pleurodynia caused by enteroviruses is characterized by severe paroxysmal pain in the chest and abdomen, along with fever, and sometimes nausea, headache, and emesis. Pericarditis and/or myocarditis are typically caused by enteroviruses. Arrythmias, heart failure, and myocardial infarction have also been reported. Acute hemorrhagic conjunctivitis can be caused by enteroviruses.

Enterovirus infection may cause hand, foot and mouth disease (HFMD). HFMD is a common, self-limiting childhood illness most commonly caused by infection by Coxsackievirus A16 (CV-A16) virus or Enterovirus EV-A71, but also other Enterovirus A serotypes such as CV-A2, CV-A4, CV-A5, CV-A6, CV-A7 and CV-A10, may cause hand, foot and mouth disease, and in addition CV-B1, CV-B2 and CV-B5 may cause HFMD (Li, et al., The Characteristics of Blood Glucose and WBC Counts in Peripheral Blood of Cases of Hand Foot and Mouth Disease in China: A Systematic Review. PLoS ONE 7(1): e29003; published Jan. 3, 2012).

However, Enterovirus 71 (EV-A71) and Coxsackievirus A16 (CV-A16) are the Enterovirus serotypes notable as the major causative agents for HFMD, but in addition EV-A71 is frequently also associated with severe central nervous system complications and in some cases cardiovascular system manifestations. EV-A71 was first isolated and characterized from cases of neurological disease in Califomia in 1969. To date, little is known about the molecular mechanisms of host response to EV-A71 infection, but increases in the level of mRNAs encoding chemokines, proteins involved in protein degradation, complement proteins, and pro-apoptotic proteins have been implicated.

Over the past decade and a half, HFMD has emerged as a worldwide public health problem particularly in the Asia-Pacific region wherein the disease is caused by a group of non-polio enteroviruses of the Picornaviridae family of which Coxsackievirus A16 (CV-A16) and Enterovirus 71 (EV-A71) are the most common etiological agents. Fatal EV-A71 were first seen in Sarawak, Malaysia in 1997 followed by a large outbreak in Taiwan in 1998 and then annually in one or another country in the Asia Pacific. A huge EV-A71 outbreak was seen in China in 2008 and this disease was made notifiable in China and other countries. According to the World Health Organization (WHO) situational update (dated 11 Dec. 2013), for the year of 2013 there were 1,651,959 cases with 265 deaths reported in China, 71,627 cases with 19 deaths reported in Vietnam and 294,535 cases reported in Japan amongst other countries reflecting the profound extent of the HFMD disease according to the WHO. In May 2013, 5 cases of polio-like paralysis (1 case in Victoria) with 27 severe EV-A71-associated cases were reported in New South Wales, Australia as a result of an EV-A71 strain that has been circulating in Asia for some time and was only detected recently in Australia, implicating global travel playing a key role in facilitating disease transmission. In China where the majority of HFMD cases are seen, there continues to be regular outbreaks peaking in May and June from 2009 through 2015.

In HFMD, enteroviruses are excreted in feces and are also found in pharyngeal secretions. Transmission is associated with close contact among children and through environmental contamination. Disease is characterized by an acute onset of fever with a rash on the palms, soles, buttocks, and knees, and vesicles on buccal membranes that usually resolve in 7-10 days. However, a small proportion of children with HFMD develop severe central nervous system disease which is often fatal.

Severe HFMD disease involving primarily the neurologic and cardiovascular systems manifesting as syndromes such as meningitis, encephalitis, acute flaccid paralysis, pulmonary edema and cardiac failure generally occur only with EV-A71 infection. In the Asia-Pacific Region the most devastating neurological syndrome is brainstem encephalitis, which has a mortality rate of 40-80 percent. Children with severe HFMD may take months to recover, and in some cases the neurologic damage may be permanent. Currently, there is no specific antiviral treatment for HFMD.

With regard to vaccine development, there are numerous publications describing approaches to prepare novel vaccines directed against Enterovirus A serotypes such as EV-A71 and CV-A16, and especially against EV-A71. Three companies in China, as the country most affected by large EV-AV71 outbreaks, have completed phase III trials of an inactivated whole virus EV-A71 monovalent vaccine. The major drawback with such vaccines is risk of infection due to incomplete inactivation, as well as environmental risks during production.

Subunit protein vaccines based especially upon the EV-A71 VP1 protein have been tried in academic settings without progressing to more commercial development. A good example is from W U, et al., (Vaccine 20, 895-904 (2002)) where a VP1 subunit vaccine was immunogenic and elicited neutralizing antibodies, but was inferior to an inactivated whole virus vaccine control in both titre and duration of effect, with in vivo protection only seen with low titre virus challenge and not a high titre virus challenge, compared to an inactivated virus vaccine.

In addition, more advanced state-of-the-art technologies have been used to develop prototype vaccines and vaccines in early preclinical stages for both EV-A71 and CV-A16.

The use of VLPs in vaccine formulations has been investigated. Virus-like particles of Enterovirus EV-A71 are described by CHUNG, et al. (2006) (World J Gastroenterol 12(6): 921-927, 2006), CHUNG, et al. (2008) (Vaccine 26:1855-1862, 2008), and CHUNG, et al. (2010) (Vaccine 28:6951-6957, 2010) wherein Enterovirus EV-A71 VLPs consist of EV-A71 structural capsid polypeptides VP0, VP1 and VP3.

Furthermore, the present applicant's earlier published patent application; International Publication Number WO 2013/098655, discloses EV-A71, CV-A16 and poliovirus VLPs comprising VP0 polypeptides, VP1 polypeptides, VP2 polypeptides, VP3 polypeptides and VP4 polypeptides.

Since both EV-A71 and CV-A16 co-circulate and cause HFMD with approximately equal frequency in South East Asia, vaccination against both pathogens is needed for protection against clinical HFMD diseases. The phase III trials already conducted in China with the monovalent inactivated whole virus Enterovirus EV-A71 vaccines show very clearly that there is no cross protection against non EV-A71 enteroviruses as shown in ZHU, et al., (The Lancet, 381:2024-2032, 2013); ZHU, et al. (NEJM 370(9):818-828, 2014) and L I, et al. (NEJM 370(9):829-837, 2014).

There is a strong need for vaccines against enteroviruses causing neurological disease, such as EV-A71. Thus, vaccines directed against Enterovirus EV-A71 and Enterovirus CV-A16 are strongly needed.

Thus, it is of utmost importance to develop effective monovalent EV-A71 and CV-A16 vaccines which may be used separately or together for vaccination against Enterovirus EV-A71 and Enterovirus CV-A16 infection.

It is indeed well recognized in the vaccine art, that it is unclear whether an antigen derived from a pathogen will elicit protective immunity. ELLIS (Chapter 29 of Vaccines, PLOTKIN, et 1a. (eds) WB Saunders, Philadelphia, at page 571, 1998) exemplifies this problem in the recitation that “the key to the problem (of vaccine development) is the identification of that protein component of a virus or microbial pathogen that itself can elicit the production of protective antibodies, and thus protect the host against attack by the pathogen.”

Prior antigenic compositions which elicit antibodies directed against Enterovirus CV-A16 have been reported in the literature; however, no vaccine is currently available.

VLP vaccines have the advantages of being immunogenic, noninfectious, and are accessible to quality control as well as to scaling-up during production. It is understood in the art that VLPs are noninfectious for the fact that VLPs do not comprise a genome. Enterovirus VLPs do not comprise an RNA genome.

Current development of vaccines directed against CV-A16 has focused on production of virus-like particles of Enterovirus CV-A16 obtained by utilizing baculovirus-insect cell expression systems.

The expression systems known in the art for producing Enterovirus CV-A16 VLPs rely on cleavage of the CV-A16 P1 polypeptide by a CV-A16 3CD protease to provide the capsid structural polypeptides which self-assemble into VLPs. See, for example, LIU, et al. (A virus-like particle vaccine for coxsackievirus A16 potently elicits neutralizing antibodies that protect mice against lethal challenge. Vaccine. 30 (2012) 6642-6648), Ku, et al. (Vaccine 32:4296-4303, 2014) and GONG, et al. (J. Virol. 88:6444-52, 2014).

When CV-A16 virus-like particles were produced in baculovirus and used to immunize mice, neutralization titres were reported to be in the wide range from 40 to 320 (GONG, et al.), 512 to 8192 (K U, et al.) and from 1600 to 32000 (LIU, et al.). Such a wide range of titres from different studies, and especially from different studies within the same group (K U, et al. and LIU, et al.) presents a heterogeneity in response which suggests that the product VLPs of CV-A16 obtained by are heterogeneous.

Indeed, baculovirus expression in insect cells by co-expression of the CV-A16 P1 polypeptide and the CV-A16 3CD protease provides compositions having two distinct populations of products, as shown by the sedimentation profile on sucrose gradients and Western blotting. (LIU. et al., Vaccine 30:6642-48, 2012). Such CVA-16 VLPs must be partially purified by fractionation to yield a homogeneous preparation of VLPs to be used for immunization.

With regard to the observed heterogeneity in immune responses elicited by those VLP preparations reported in the literature, one possible explanation for these results is that the VLP production leads to aggregation of VLPs.

It is known in the art that VLPs have the propensity to aggregate into high molecular weight entities. The biological significance of VLP aggregation, for example, on antigenicity and immunogenicity, is unclear.

With regard to vaccine development, aggregation of the VLPs present enormous challenges during production, such as during purification and formulation. For example, it is not possible to titer VLP products which comprise aggregated VLPs and, therefore, these products cannot be used for vaccination and are not allowed by regulatory authorities. Thus, aggregation of the VLPs poses challenges to downstream processes and subsequent vaccine approval.

The consistency of the VLP antigens is of utmost importance for vaccine manufacture to ensure vaccine quality and efficacy.

The instant invention provides methods for making VLPs and compositions enriched in CV-A16 VLPs which exhibit conformational epitopes which elicit immune responses and/or neutralizing antibody responses which may be used in vaccines directed against Enterovirus CV-A16.

The compositions obtained by the instant method utilizing the instant expression cassette, whereby a CV-A16 P1 polypeptide is processed into structural polypeptides by an EV-A71 3CD protease, exhibit a single population of VLPs which elicit an immune response and/or neutralizing antibody response. The compositions are essentially free of aggregates of VLPs.

The instant methods described herein reduce VLP aggregation and improve functional VLP yields.

Moreover, it has never been shown that Enterovirus VLPs may form by utilizing an expression cassette for expression of an Enterovirus P1 polypeptide and an EV-A71 3CD protease, wherein the P1 polypeptide is derived from an Enterovirus which is not EV-A71. What is more, it has not been shown that any such cleavage of the Enterovirus P1 may be expected to provide structural capsid polypeptides which assemble to form VLPs and, moreover, that conformational epitopes are preserved on the surface of the VLP such that the VLPs may elicit protective and/or neutralizing immune responses directed against the Enterovirus from which the P1 polypeptide originates.

It is surprising that an EV-A71 3CD protease may process an Enterovirus CV-A16 P1 polypeptide into its cognate structural capsid polypeptides and, moreover, that such structural capsid polypeptides would assemble to provide a VLP which provides immune responses and/or neutralizing antibody responses such that the VLPs may be used in a vaccine directed against Enterovirus CV-A16.

Moreover, it is unexpected that processing of the Enterovirus CV-A16 P1 polypeptide into structural capsid polypeptides by an Enterovirus EV-A71 3CD protease would provide VLPs which do not exhibit significant aggregation and which may be used in a vaccine directed against Enterovirus CV-A16.

The instant invention, therefore, solves the problems associated with developing Enterovirus VLP vaccines, for example, unwanted aggregation of VLPs and lower immunogenicity.

The VLPs of the instant invention may be characterized as providing unexpectedly improved immune responses when utilized as a vaccine, which immune responses may be more consistent and predictable compared those obtained with prior art VLP preparations.

The instant invention provides improved methods of producing Enterovirus virus-like particles and compositions comprising virus-like particles and essentially free of VLP aggregation or aggregates, and which VLPs elicit effective immune responses and/or neutralizing antibody responses such that the VLPs may be used in a vaccine directed against Enterovirus CV-A16.

### SUMMARY OF THE INVENTION

A Virus-Like Particle (VLP) which elicits an immune response and/or neutralizing antibody response against infection by the Enterovirus from which the Enterovirus structural polypeptides of the VLP are derived; such a

VLP wherein the Enterovirus is selected from Enterovirus A, Enterovirus B, Enterovirus C and Enterovirus D; such a

VLP which is formed from CV-A16 P1 polypeptides which are processed by an EV-A71 3CD protease, which VLP is characterized by improved immunogenicity over VLPs which are not formed by an EV-A71 3CD protease, such a

VLP wherein the Enterovirus is CV-A16; such a

VLP wherein the Enterovirus is selected from Poliovirus 1, Poliovirus 2 and Poliovirus 3; such a

nucleic acid encoding an expression cassette comprising a promoter operably linked to a nucleic acid encoding an Enterovirus P1 polypeptide comprising structural polypeptides VP0, VP3 and VP1, which is operably linked to a nucleic acid encoding an Internal Ribosome Entry Site (IRES), and which is operably linked to a nucleic acid encoding an EV-A71 3CD protease; such a

nucleic acid encoding the expression cassette, wherein the Enterovirus P1 polypeptide is from an Enterovirus selected from Enterovirus A, Enterovirus B, Enterovirus C and Enterovirus D; such a

nucleic acid encoding the expression cassette, wherein the Enterovirus P1 polypeptide is from Enterovirus CV-A16; such a

nucleic acid encoding the expression cassette, wherein the Enterovirus P1 polypeptide is from an Enterovirus selected from Poliovirus 1, Poliovirus 2 and Poliovirus 3; such a

nucleic acid encoding the expression cassette, wherein the EV-A71 3C or 3CD protease in under translational control of the IRES; such a

nucleic acid encoding the expression cassette, wherein the nucleic acid sequence encoding the IRES has been genetically modified; such a

nucleic acid encoding the expression cassette, wherein the nucleic acid sequence encoding the 3CD has been genetically modified; such a

nucleic acid encoding the expression cassette, wherein the IRES is derived from Encephalomyocarditis virus (EMCV) or an Enterovirus; such a

method of making an Enterovirus VLPs comprising the step of culturing a prokaryotic or eukaryotic host cell comprising a nucleic acid encoding an expression cassette comprising a promoter operably linked to a nucleic acid encoding an Enterovirus P1 polypeptide comprising structural polypeptides VP0, VP3 and VP1, which is operably linked to a nucleic acid encoding an Internal Ribosome Entry Site (IRES), and which is operably linked to a nucleic acid encoding an EV-A71 3CD protease, for a period of time sufficient to produce Enterovirus P1 polypeptides and EV-A71 3CD proteases, and to form VLPs; such a

method further comprising the step of recovering the VLPs from the host cell; such a

composition comprising Enterovirus CV-A16 VLPs made by the method, wherein the composition is essentially free of aggregates of Enterovirus CV-A16 VLPs; such a

vaccine comprising VLPs which elicits an immune response and/or neutralizing antibody response against infection by the Enterovirus from which the Enterovirus polypeptides of the VLP are derived; such a

vaccine comprising VLPs which elicits an immune response and/or neutralizing antibody response against infection by the Enterovirus from which the Enterovirus polypeptides are derived essentially free of aggregation; such a

vaccine comprising VLPs which elicits an immune response and/or neutralizing antibody response against infection by the Enterovirus from which the Enterovirus polypeptides are derived essentially free of aggregates; such a

vaccine comprising VLPs which elicits an immune response and/or neutralizing antibody response against infection by the Enterovirus from which the Enterovirus polypeptides are derived essentially free of aggregates of VLPs; such a

vaccine wherein the Enterovirus is selected from Enterovirus A, Enterovirus B, Enterovirus C and Enterovirus D; such a

vaccine wherein the Enterovirus is CV-A16; such a

vaccine wherein the Enterovirus is selected from Poliovirus 1, Poliovirus 2 and Poliovirus 3; such a

vaccine comprising the VLPs obtained by the method; such

VLPs for use in a vaccine for vaccinating a subject against infection by the Enterovirus, the use comprising administering to the subject the Enterovirus Virus-Like Particle in an amount effective to elicit an immune response and/or neutralizing antibody response directed against the Enterovirus when administered to the subject; such a

method of providing an immune response and/or neutralizing antibody response against infection by an Enterovirus in a subject, the method comprising administering to the subject the Virus-Like Particles in an amount effective to provide such immune response and/or neutralizing antibody response against infection by an Enterovirus in the subject.

### BRIEF DESCRIPTION OF THE DRAWINGS

FIG. 1. A schematic representation of the elements of the expression cassettes for recombinant baculovirus expression constructs to provide Enterovirus CV-A16 VLPs. Enterovirus expression cassettes [P1+IRES+3CD]. A. Expression construct SXT6, the expression cassette encoding an Enterovirus CV-A16 P1 polypeptide, an IRES, and an Enterovirus EV-A71 3CD protease. B. Expression construct SXT9, the expression cassette encoding an Enterovirus CV-A16 P1 polypeptide, an IRES, and an Enterovirus CV-A16 3CD protease.

FIG. 2. Size-exclusion analysis of Enterovirus CV-A16 VLPs produced using recombinant baculovirus comprising an expression cassette comprising an Enterovirus CV-A16 P1 polypeptide and an Enterovirus CV-A16 3CD protease.

FIG. 3. Size-exclusion analysis of Enterovirus CV-A16 VLPs produced using recombinant baculovirus comprising an expression cassette comprising an Enterovirus CV-A16 P1 polypeptide, an IRES and an Enterovirus EV-A71 3CD protease.

FIG. 4. Western blots showing that Enterovirus CV-A16 structural polypeptides VP0, VP1 and VP3 (arrows) are present in the lysates of the Sf9 cells infected with the recombinant baculovirus expression construct SXT6, wherein the expression cassette comprises the coding sequence of CV-A16 P1 polypeptide and an EV-A71 3CD protease. FIG. 4A was probed with mouse monoclonal antibody against Enterovirus A VPONP2; FIG. 4B was probed with rabbit hyperimmune polyclonal antibody against VP1; and FIG. 4C was probed with rabbit hyperimmune polyclonal antibody against VP3. Arrows point to the capsid proteins VP0, VP1 and VP3 in FIGS. 4A, 4B and 4C, respectively.

FIG. 5. Indirect ELISA showing that antibodies directed against Enterovirus CV-A16 virus antigens were produced when mice were immunized with virus-like particles as immunogens. The level of antibodies elicited by VLPs produced by recombinant baculoviruses comprising an expression cassette comprising CV-A16 P1 polypeptide, an IRES and an EV-A71 3CD protease (SXT6) is compared to the level of antibodies elicited by VLPs produced by recombinant baculoviruses comprising an expression cassette comprising CV-A16 P1 polypeptide, an IRES and a CV-A16 3CD protease (SXT9). Sera from mice immunized with SXT6 VLPs or SXT9 VLPs bind to CV-A16 antigens from CV-A16 infected rhabdomyosarcoma cell lysates. The solid square symbols represent individual sera from the control mice immunized with control antigen, FGUS. The solid circles represent individual sera from the mice immunized with SXT6 VLPs. The open squares represent individual sera from the mice immunized with SXT9 VLPs. The Y axis represents the net optical density readings at a 450 nm wavelength (OD₄₅₀).

FIG. 6. EV-A71 VLP expression cassette [P1+IRES+3CD] in the pSN01 baculovirus expression construct described in PCT/IB2012/003114 comprising an Enterovirus EV-A71 P1, an IRES and an EV-A71 3CD protease.

### DETAILED DESCRIPTION OF THE INVENTION

The instant invention provides a method for producing Enterovirus virus-like particles and compositions which are improved over those compositions comprising Enterovirus virus-like particles obtained by means known in the art.

The invention in an aspect includes a method for production of Enterovirus virus like particles (VLPs) which may include the steps of: (i) constructing an expression cassette comprising a promoter operably linked to a nucleic acid encoding an Enterovirus P1 polypeptide which is/are operably linked to an internal ribosome entry site (IRES), which IRES is operably linked to an Enterovirus 3C or 3CD protease; (ii) transfecting or transforming a suitable host cell with the expression cassette; (iii) culturing the host cells under conditions in which VLPs are produced by the cell after expression of the nucleic acids comprised in the cassette.

A nucleic acid or recombinant DNA molecule may be obtained whereby open reading frames which encode human Enterovirus, including Coxsackievirus A16, EV-A71, and/or EV 68, polypeptides and proteases may be amplified by PCR amplification using suitably designed primers complementary to nucleic acid sequences of said enteroviruses. Suitable primers may be designed according to standard techniques from publicly available nucleic acid sequences of enteroviruses. Moreover, nucleic acid sequences may be synthesized de novo according to technologies known in the art. Complete genome sequences are available in GenBank and are accessible at the National Center for Biotechnology Information (NCBI).

In an embodiment, an Enterovirus CV-A16 P1 polypeptide may be expressed as a polypeptide which is subsequently cleaved by an Enterovirus EV-A71 3C or 3CD protease into VP0, VP1 and VP3 virus polypeptides, or immunologically or biologically active fragments thereof, which Enterovirus polypeptides elicit neutralizing antibodies directed against enteroviruses. The VP0 protein may be further cleaved into VP2 and VP4 proteins, or immunologically or biologically active fragments thereof which elicit neutralizing antibodies directed against enteroviruses. The virus polypeptides self-assemble into VLPs. Further it will be appreciated that the protease genes may be included in the same DNA recombinant molecule of the VLP expression cassette or in different DNA recombinant molecules, and/or expressed from different promoters or translation elements.

Recombinant DNA molecules and nucleic acids of the VLP expression cassettes may be devised whereby open reading frames which encode human Enterovirus CV-A16 structural proteins and human Enterovirus EV-A71 3CD proteases may be obtained by PCR amplification using suitably designed primers complementary to nucleic acid sequences of human enteroviruses or may be synthesized de novo according to technologies known in the art.

The present invention encompasses a VLP expression cassette which harbors the gene sequences for Enterovirus structural capsid proteins (i.e., the P1 region) with a protease (3CD) which is necessary for the processing of the P1 polypeptide into the individual polypeptides of the virus capsid, thus allowing the self-assembly of the structural capsid polypeptides into Enterovirus VLPs. The expression cassette is a bicistronic vector which uses a promoter upstream of the nucleic acid coding sequence for an Enterovirus P1 polypeptide. Downstream from the cistron encoding the P1 polypeptide is an internal ribosome entry site (IRES) sequence followed by the cistron containing a nucleotide sequence encoding an Enterovirus EV-A71 3CD protease.

Expression of the P1 polypeptide and the 3CD protease proceeds from a single bicistronic message wherein the 3CD protease gene is translated in a cap-independent fashion under the translational control of the IRES.

For example, the expression cassette of the invention may comprise a promoter which is operably linked to a nucleic acid encoding a human Enterovirus A P1 polypeptide, an IRES, and a human Enterovirus EV-A71 3CD protease.

In an embodiment, the expression cassette of the invention may comprise a promoter which is operably linked to a nucleic acid encoding a human Enterovirus CV-A16 P1 polypeptide, an IRES, and a human Enterovirus EV-A71 3CD protease.

A bicistronic vector is constructed in which a plasmid contains a polyhedrin promoter upstream of the coding sequence for the Enterovirus P1 polypeptide. Downstream from the cistrons encoding Enterovirus P1 is an Encephalomyocarditis virus (EMCV) internal ribosome entry site (IRES) sequence (GenBank accession number AF113968.2; nucleotidesl 666 to 2251) followed by the cistrons containing the nucleotide sequence encoding the Enterovirus EV-A71 3CD protease.

In an embodiment, the expression cassette of the invention may comprise a promoter which is operably linked to a nucleic acid encoding a human Enterovirus CV-A16 P1 polypeptide, an EMCV IRES, and a human Enterovirus EV-A71 3CD protease.

Baculovirus expression utilizing an expression cassette comprising a human Enterovirus CV-A16 P1 polypeptide, an IRES, and a human Enterovirus EV-A71 3CD protease provides functional virus-like particles which elicit protective and/or neutralizing immune responses.

It is surprising that a 3CD protease derived from Enterovirus EV-A71 instead of an Enterovirus CV-A16 3CD protease for cleavage of the Enterovirus CV-A16 P1 polypeptide resulted in virus-like particles.

Virus-like particles of Enterovirus CV-A16 and methods for producing VLPs are known in the art. For example, LIU, et al., K U, et al. (Vaccine 32:4296-4303, 2014) and GONG, et al. (J. Virol. 88:6444-52, 2014) describe formation of VLPs by co-expression of an Enterovirus CV-A16 P1 polypeptide and an Enterovirus CV-A16 3CD protease in a host cell. When the virus-like particles are administered to mice in conjunction with alum as adjuvant, neutralization titres were reported to be in the wide range from 40 to 320 (GONG, et al.), from 512 to 8192 (K U, et al.) and from 1600 to 32000 (LIU. et al.). Such a wide range of titres from different studies presents a heterogeneity in response which warrants further investigation.

Similar to the methods disclosed in the above-cited art, the applicant produced Enterovirus CV-A16 virus-like particles in a baculovirus expression system, where a CV-A16 P1 polypeptide is cleaved by a CV-A16 3CD protease in the host cell, and the host cell lysates were obtained for analysis and comparison with the Enterovirus CV-A16 virus-like particles obtained according to the present invention, where the CV-A16 virus-like particles were produced by cleavage of the CV-A16 P1 polypeptide by a EV-A71 protease.

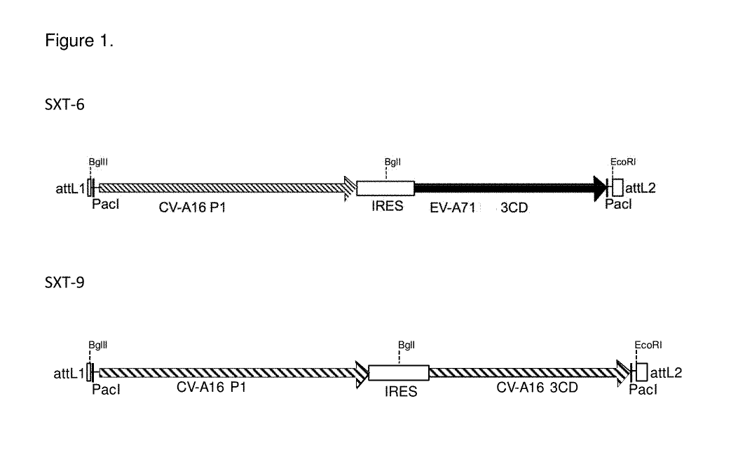

With regard to the instant method for producing Enterovirus CV-A16 VLPs, and in contrast to the foregoing, insect cells were infected with a baculovirus harboring an expression cassette, SXT-6, as depicted in FIG. 1. The expression cassette comprises a promoter which is operably linked to a nucleic acid encoding a CV-A16 P1 polypeptide, an IRES, and an EV-A71 3CD protease, wherein the EV-A71 3CD protease is under the translational control of the IRES. In the infected host cells, the CV-A16 P1 polypeptide is cleaved by the EV-A71 3CD protease into structural capsid polypeptides and which capsid polypeptides assemble into functional virus-like particles which elicit protective and/or neutralizing immune responses.

The host cell lysates comprising virus-like particles produced by the methods disclosed in the art and host cell lysates obtained by the instant method were separated by size-exclusion chromatography on SEPHACRYL® S500 which separates assembled particles according to their size, the larger entities eluting from the column first. The fractions were collected and analyzed for the presence of VLPs.

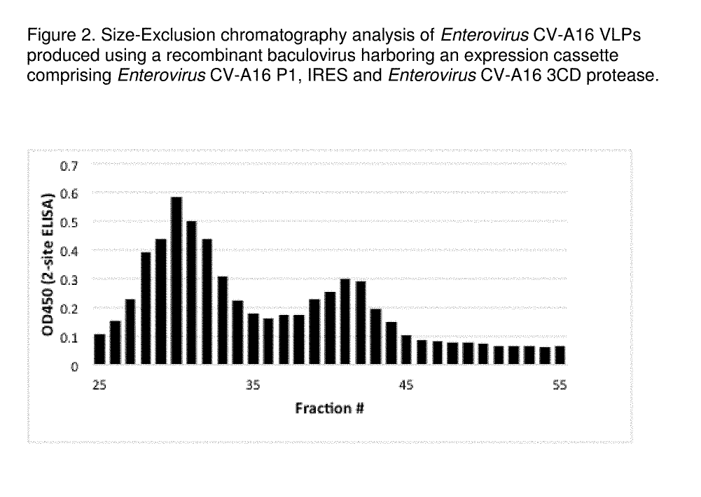

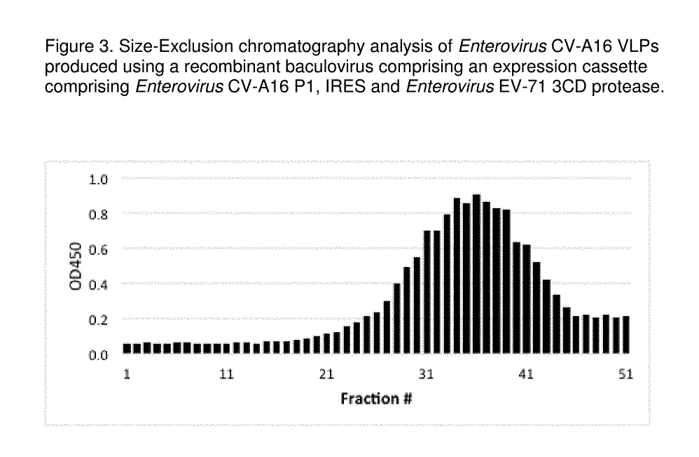

The presence of Enterovirus CV-A16 VLPs in each fraction was determined by a sandwich ELISA assay using 2 different antibodies directed against Enterovirus CV-A16 antigens to detect the presence of assembled CV-A16 polypeptides. FIGS. 2 and 3 show the results of the ELISA assay of the individual fractions obtained by the size-exclusion chromatography.

The analysis of the VLPs produced in baculovirus infected cells shows that there were clearly 2 populations of particles having different sizes produced when the Enterovirus CV-A16 P1 polypeptide was processed by the Enterovirus CV-A16 3CD protease. See FIG. 2.

Utilizing the instant method, wherein the Enterovirus CV-A16 P1 polypeptide was processed by the Enterovirus EV-A71 3CD protease, a single population of particles was produced. See FIG. 3.

These data suggest that the Enterovirus CV-A16 3CD protease processes the Enterovirus CV-A16 P1 polypeptide in a manner leading to inefficient production of virus-like particles having the correct conformation to be used in a vaccine.

Indeed, the peak on the left in FIG. 2, representing the fractions having the larger particles in the size-exclusion analysis, appears in the void volume and, thus, would consist of aggregates of assembled polypeptides. The peak on the right in FIG. 2, corresponding to fractions having smaller particles, represents VLPs which are not in aggregated form.

As shown in FIG. 2, the amount of VLPs which are in aggregated form represents a significant portion of the VLPs produced in host cells by the methods utilizing the protease natively associated with the P1 polypeptide. In vaccine development, this represents a marked loss in yield of those VLPs which may be used in an efficacious vaccine.

Aggregation of polypeptides, proteins, assembled VLPs, viruses, etc. is to be avoided in a drug or vaccine preparation for many reasons, such as a negative effect on production, purification, quality, solubility and stability. Thus, as shown in FIG. 3, the instant method of making Enterovirus CV-A16 VLPs, wherein the Enterovirus CV-A16 P1 polypeptide is processed by an Enterovirus EV-A71 3CD protease, produces a superior product in which the VLPs are not in aggregated form.

The instant method utilizing baculovirus expression of an expression cassette comprising an Enterovirus CV-A16 P1 polypeptide, an IRES, and an Enterovirus EV-A71 3CD protease reduces VLP aggregation and improves functional VLP yield, as shown by the single peak in the size-exclusion analysis of FIG. 3.

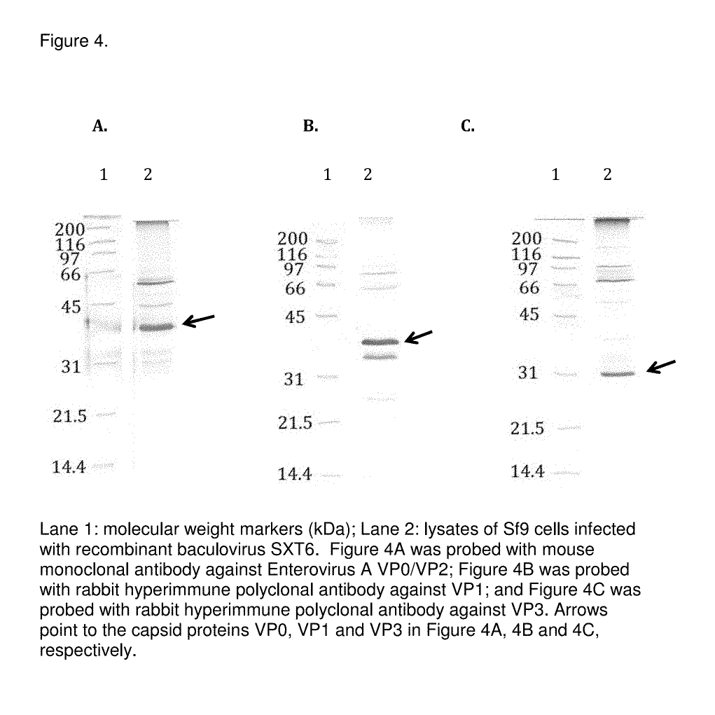

The compositions comprising VLPs produced by the instant method as described herein provide VLPs assembled from the structural capsid polypeptides. See FIG. 4 which confirms the presence of structural capsid polypeptides in the VLPs formed according to the instant methods.

The compositions comprising VLPs produced by the instant methods as described herein provide VLPs predominately of a conformation demonstrated to elicit antibodies, which antibodies are functional and able to neutralize enteroviruses, such as Enterovirus CV-A16, to high titre.

For example, the immunogenicity of Enterovirus CV-A16 VLPs produced by the methods described herein, i.e., expression from a baculovirus harboring an expression cassette comprising an Enterovirus CV-A16 P1 polypeptide and an Enterovirus EV-A71 3CD protease (SXT6 in FIG. 1) was compared to that of Enterovirus CV-A16 VLPs produced by a baculovirus harboring an expression cassette comprising an Enterovirus CV-A16 P1 polypeptide, an IRES, and an Enterovirus CV-A16 3CD protease (SXT9 in FIG. 1).

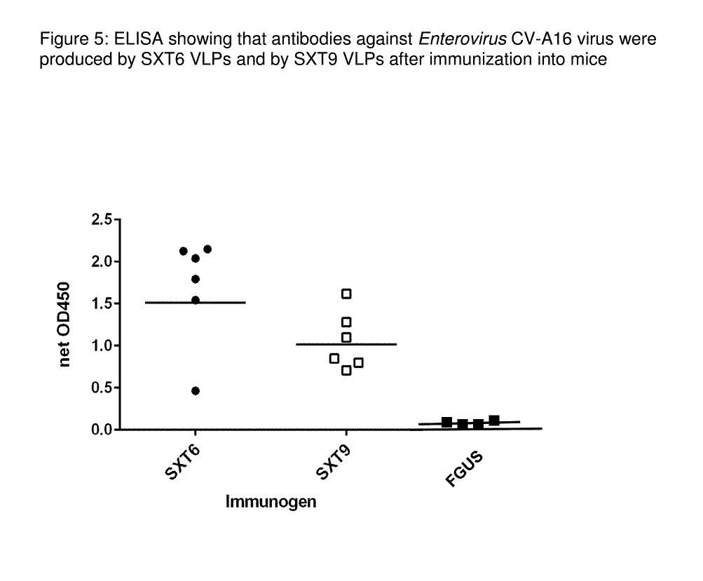

Enterovirus CV-A16 VLPs which were produced by CV-A16 3CD protease processing of CV-A16 P1 (SXT9) were immunogenic and elicited antibodies in mice. These antibodies were able to recognize Enterovirus CV-A16 virus antigens. Moreover, the Enterovirus CV-A16 VLPs which were produced by EV-A71 3CD protease processing of the CV-A16 P1 (SXT6) were immunogenic and elicited antibodies in mice. See FIG. 5.

The Enterovirus CV-A16 VLPs obtained by expression of the baculovirus expression cassette SXT6 (i.e., CV-A16 VLPs which were produced by EV-A71 3CD protease processing of the CV-A16 P1) provided an immune response which was unexpectedly improved over that immune response observed when the VLPs were obtained from the baculovirus expression cassette SXT9. See FIG. 5 which compares the immune response of the Enterovirus CV-A16 VLPs obtained by the expression cassette of baculovirus SXT6 versus the Enterovirus CV-A16 VLPs obtained by baculovirus expression utilizing the expression cassette of baculovirus SXT9.

Enterovirus CV-A16 VLPs generated by baculovirus SXT6, that is, CV-A16 VLPs which were produced by EV-A71 3CD protease processing of the CV-A16 P1, demonstrated unexpectedly improved immune responses directed against Enterovirus CV-A16 virus over VLPs obtained when CV-A16 P1 polypeptide is processed by the cognate CV-A16 3CD protease.

Such an improvement of the quality of VLPs for generating functional antibodies and improved immune responses directed against Enterovirus CV-A16 virus is not expected. The immunogenic VLPs of the instant invention may be administered to a subject to elicit superior immune responses and neutralizing antibodies directed against human enteroviruses and confer enhanced protection from infection by Enterovirus CV-A16 viruses.

The invention therefore provides virus-like particles (VLPs) for protection and/or treatment against infection by an Enterovirus. The invention further provides virus-like particles (VLPs) in the form of an immunogenic composition and/or vaccine for protection and/or treatment against infection by an Enterovirus. More specifically, the present invention provides Enterovirus CV-A16 VLPs which elicit immune responses and neutralizing antibody responses against Enterovirus CV-A16 virus infection. Even more specifically, the present invention provides for assembly of Enterovirus CV-A16 VLPs assembled from expression constructs expressing a CV-SENTINEXT 9 MY A16 P1 polypeptide and an EV-A71 3CD protease, wherein the VLPs are essentially free from aggregation.

An achievement of the present invention is to provide a novel vaccine comprising a VLP of an Enterovirus, which elicits immune responses, protective and/or neutralizing antibody responses against an Enterovirus selected from Enterovirus A, Enterovirus B, Enterovirus C or Enterovirus D and any serotype virus of these virus species, wherein the Enterovirus P1 polypeptide is processed from a protease derived from a heterologous Enterovirus, specifically Enterovirus EV-A71 3CD protease is utilized.

A further achievement of the present invention is to provide a novel vaccine comprising a VLP of Enterovirus CV-A16, which VLP elicits immune responses and/or neutralizing antibody responses against CV-A16. This achievement is more remarkable for the fact that such Enterovirus CV-A16 VLPs are assembled from the CV-A16 P1 polypeptide which has been processed by an EV-A71 3CD protease. With the instant invention, we provide for the production of a vaccine comprising VLPs eliciting one or more immune responses and/or neutralizing antibody responses to epitopes of VP0, VP1, VP2, VP3 and/or VP4 of an Enterovirus selected from Enterovirus A, Enterovirus B, Enterovirus C and Enterovirus D and any serotype virus of these virus species processed from an Enterovirus P1 polyprotein by an EV-A71 3CD protease. This is made possible by the surprising assembly of VLPs eliciting one or more neutralizing antibody epitopes of VP0, VP1, VP2, VP3 and/or VP4 of the Enterovirus and any genotype of the Enterovirus processed from such an Enterovirus P1 polyprotein by a heterologous EV-A71 3CD protease which is not natively associated with the particular P1 polypeptide.

It has surprisingly been found according to the present invention that human Enterovirus VLPs actually can assemble to mimic the antigenicity of a native virus when produced from a cassette comprising a promoter operably linked to a nucleic acid sequence encoding a Enterovirus P1 polypeptide, wherein the nucleic acid sequence encoding the Enterovirus P1 polypeptide is operably linked to a nucleic acid sequence encoding an Internal Ribosome Entry Site (IRES), wherein the nucleic acid sequence encoding the IRES is operably linked to a nucleic acid sequence encoding a heterologous EV-A71 3CD protease, wherein the heterologous EV-A71 3CD protease is under the translational control of the IRES to provide VLPs eliciting protective and/or neutralizing immune response.

Specifically, it has been found that CV-A16 VLPs according to the present invention elicit protective and/or neutralizing immune responses against Enterovirus CV-A16 infection, despite being processed by an EV-A71 3CD protease.

The invention therefore provides novel compositions and vaccine formulations comprising Enterovirus VLPs produced from an expression cassette comprising a promoter operably linked to a nucleic acid sequence encoding a Enterovirus P1 polypeptide, wherein the nucleic acid sequence encoding the Enterovirus P1 polypeptide is operably linked to a nucleic acid sequence encoding an Internal Ribosome Entry Site (IRES), wherein the nucleic acid sequence encoding the IRES is operably linked to a nucleic acid sequence encoding an EV-A71 3CD protease, wherein the EV-A71 3CD protease is under the translational control of the IRES to provide Enterovirus VLPs exhibiting neutralizing antibodies.

According to the present invention a method of preparing the VLPs of the invention is also provided.

Thus, the invention in an additional aspect includes a method for production of the VLPs of the invention, which may include the steps of: constructing an expression cassette comprising a promoter operably linked to a nucleic acid encoding a Enterovirus P1 polypeptide, wherein the nucleic acid encoding the Enterovirus P1 polypeptide is operably linked to a nucleic acid encoding an Internal Ribosome Entry Site (IRES), wherein the nucleic acid encoding the IRES is operably linked to a nucleic acid encoding an EV-A71 3CD protease, wherein the EV-A71 3CD protease is under the translational control of the IRES to provide Enterovirus VLPs exhibiting an immune response and/or neutralizing antibody response.

Expression cassettes cloned into suitable vectors, such as for example baculovirus vectors, and transformed/transfected into appropriate host cells, such as for example insect cells such as Spodoptera frugiperda (e.g. Sf9 cells) for expression and purification of the VLPs of the invention are provided.

The invention in an additional aspect includes a method for production of the VLPs, which method may include the steps of: (i) constructing an expression cassette comprising a promoter operably linked to a nucleic acid which encodes a Enterovirus polypeptide P1, which nucleic acid is operably linked to an internal ribosome entry site (IRES), which IRES is also operably linked to a nucleic acid encoding an Enterovirus EV-A71 3C or 3CD protease; (ii) transfecting, transforming or infecting a suitable host cell with a construct containing the expression cassette; (iii) culturing the host cells under conditions in which virus like particles (VLPs) are produced by the cell after expression of the nucleic acids comprised in the cassette.

Making truncations and mutations of the 3CD protease in the expression cassette may achieve increased yield of VLPs. For example, the Glycine of the EV-A71 3C protease, which is amino acid 1671 of GenBank accession number DQ341362.1 may advantageously be changed to an Alanine (G1671A) using site directed mutagenesis for the expression of mutant EV-A71 3C and subsequent processing of an Enterovirus P1 polypeptide.

Expression cassettes cloned into vectors, such as for example baculovirus vectors, and transformed, transfected or infected into appropriate host cells, such as for example insect cells, such as but not limited to Spodoptera frugiperda (e.g. Sf9 cells), for expression and purification of the VLPs of the invention are provided.

Pharmaceutically useful compositions comprising the VLPs of the invention may be formulated according to known methods such as by the admixture of pharmaceutically and immunologically acceptable carriers and/or adjuvants and/or additional antigenic determinants. Examples of such carriers and methods of formulation may be found in Remington's Pharmaceutical Sciences. To form a pharmaceutically acceptable composition suitable for effective administration, such compositions will contain an effective amount of one or more of the VLPs of the invention. Such compositions may contain VLPs derived from more than one type of Enterovirus.

Vaccine compositions of the invention may be administered to an individual in amounts sufficient to elicit immune responses and/or neutralizing antibody responses directed against one or more Enterovirus. The effective amount may vary according to a variety of factors such as the individual's condition, weight, sex and age. Other factors include the mode of administration. The vaccines may be provided to the individual by a variety of routes such as subcutaneous, topical, oral, mucosal, intravenous, parenterally, and intramuscular.

The vaccines comprising one or more of the VLPs of the invention may contain additional antigenic determinants and/or adjuvants well known in the art necessary to elicit a protective and/or neutralizing immune response in the host. Such vaccines are generally safe, and do not have toxic side effects; and may be administered by an effective route; and are stable; and are compatible with vaccine carriers known in the art.

The vaccine may be administered in dosage forms known in the art such as for example, but not limited to, a form for injection, capsules, suspensions, elixirs, or liquid solutions. The vaccine may be administered in single or multiple doses. The invention in another aspect includes one or more of the VLPs of the invention in combination with one or more suitable adjuvants such as ISCOMS, alum, aluminum hydroxide, aluminum phosphate, Quil A and other saponins or any other adjuvant as described, for example, in Vanselow (1987) S. Vet. Bull. 57 881-896. The meaning of the terms “aluminum phosphate” and “aluminum hydroxide” as used herein includes all forms of aluminum phosphate or aluminum hydroxide, which are suitable for adjuvanting vaccines.

As used herein and in the claims, the terms and phrases set out below have the meanings which follow.

“Antibody” refers to an immunoglobulin molecule produced by B lymphoid cells with a specific amino acid sequence evoked in humans or other animals by an antigen (immunogen). These molecules are characterized by reacting specifically with the antigen.

“Antibody response” or “humoral response” refers to a type of immune response in which antibodies are produced by B lymphoid cells and are secreted into the blood and/or lymph in response to an antigenic stimulus. In a properly functioning immune response, the antibody binds specifically to antigens on the surface of cells (e.g., a pathogen), marking the cell for destruction by phagocytotic cells and/or complement-mediated mechanisms.

“Antigen” refers to any substance that, as a result of coming in contact with appropriate cells, induces a state of sensitivity and/or immune responsiveness and that reacts in a demonstrable way with antibodies and/or immune cells of the sensitized subject in vivo or in vitro.

“Epitope” refers to the simplest form of an antigenic determinant, on a complex antigen molecule. This is the specific portion of an antigen that is recognized by an immunoglobulin or T-cell receptor.

“Cellular response” or “cellular host response” refers to a type of immune response mediated by specific helper and killer T-cells capable of directly eliminating virally infected or cancerous cells.

“Antigen-presenting cell” refers to the accessory cells of antigen inductive events that function primarily by handling and presenting antigen to lymphocytes. The interaction of antigen presenting cells (APC) with antigens is an essential step in immune induction because it enables lymphocytes to encounter and recognize antigenic molecules and to become activated. Exemplary APCs include macrophages, Langerhans-dendritic cells, Follicular dendritic cells, and B cells.

“B-cell” refers to a type of lymphocyte that produces immunoglobulins or antibodies that interact with antigens.

“Cytotoxic T-lymphocyte” is a specialized type of lymphocyte capable of destructing foreign cells and host cells infected with the infectious agents which produce viral antigens.

“Essentially free of aggregation or aggregates” when used in this specification to describe the VLPs, the vaccines and the compositions means that the VLPs, the vaccines or the compositions comprise less than 10%, less than 5%, less than 2%, or less than 1% of aggregation or aggregates.

The language “consisting essentially of” or “consists essentially of” means that in addition to those components which are mandatory, other components may also be present in compositions, provided that the essential, basic and/or novel characteristics of the compositions are not materially affected by their presence.

Bivalent: Bivalent when used to describe a vaccine means that the vaccine will elicit an immune response against two Enteroviruses.

Heterologous: Heterologous, as pertaining to Enteroviruses, are two or more Enteroviruses which belong to different families, species, serotypes, genogroups or strains. Heterologous, as pertaining to proteases, means that the protease derives from an Enterovirus which is different from the Enterovirus from which the P1 polypeptide derives (i.e., derives from a heterologous Enterovirus). An Enterovirus P1 polypeptide is processed by a protease which is not natively associated with the 3CD protease (i.e., not the cognate protease).

Neutralizing antibody response: A neutralizing antibody response is an immune response wherein specialized cells of the immune system recognize the presentation of antigen(s), and launch a specific immune response, which prevents infection of target cells from an agent, for example a virus.

In an embodiment, the VLPs according to the invention can induce an immune response. The term “immune response” and/or “neutralizing antibody response” as used herein is intended to mean that the vaccinated subject may resist or protect itself against an infection with the pathogenic agent against which the vaccination was done.

Operably linked: Operably linked means that the components described are in a relationship permitting them to function in their intended manner. Thus, for example, a promoter “operably linked” to a nucleic acid means that the promoter and the nucleic acid of a cistron, or more than one cistron, are joined in such a manner that a single cistronic, a single bicistronic, or a single multicistronic messenger RNA (mRNA) may be produced. Protein expression of the messenger RNA may be regulated according to transcriptional/translational elements of the promotor and/or nucleic acid. In another example, an Internal Ribosome Entry Site (IRES) sequence, which is inserted into an expression cassette in an orientation, which is upstream (5′) to a cistron means that the IRES sequence and the nucleic acids of the cistron are joined in such a manner that downstream of the IRES, translation of the cistronic mRNA is regulated under the control of the IRES.

Virus-like particle: A virus-like particle is an assembly of viral structural polypeptides, i.e. a combination of the capsid polypeptides VP0, VP1, VP2, VP3, VP4, which capsid polypeptides/proteins assemble in a manner and conformation similar to the authentic virus structurally, however, the VLPs do not comprise a virus genome.

The particle may be characterized by the hallmarks of a native capsid.

Picornavirus capsid structure is characterized by a five-fold vertex surrounded by a canyon in which a pocket factor is located which stabilizes the capsid structure. When the virus interacts with its cellular receptor, the binding of the receptor is often to a wall of the canyon, displacing the pocket factor and causing a structural change causing the virus to form what is called the A particle wherein there are holes formed in the capsid which allow the virus genome to escape into the cell. ROSSMANN, et al., (Trends in Microbiology (2002) vol 10 No 7 324-331) reviews the Picornavirus-receptor interactions very well. Thus, an antibody which blocks the interaction of the capsid to the virus receptor will function as a neutralizing antibody and block infection.

EV-A71 has neutralizing epitopes which are located elsewhere and not on the five-fold vertex as shown in PLEVKA, et al. (PNAS 111(6):2134-9 (2014)). There are neutralizing epitopes on the two-fold and three-fold axes involving interactions between VP3 and VP2.

Enterovirus P1: An Enterovirus P1 polypeptide is the primary structural polypeptide of an Enterovirus from which individual structural polypeptides VP0, VP1, VP2, VP3 and VP4 may be cleaved. The order in which the structural polypeptides are arranged on the P1 polypeptide, starting from the N-terminus, is VP0, VP3 and VP1. During encapsidation of the RNA genome in the native virus, VP0 is cleaved into polypeptides VP4 and VP2.

Essentially free: means that compositions are more than 90% free of aggregates of VLPs. An embodiment may provide a composition more than 95% free of aggregates of VLPs. Moreover, an embodiment may provide for a composition more than 99% free of aggregates of VLPs.

In an embodiment, the expression cassette consists essentially of a nucleic acid encoding a human Enterovirus A P1 polypeptide, an IRES, and a human Enterovirus 3CD protease derived from a different species/genotype, wherein the 3CD protease is under the translational control of the IRES, and which 3CD protease processes the human Enterovirus A P1 polypeptide into structural capsid polypeptides. The structural capsid polypeptides self-assemble into virus-like particles.

In an embodiment, the expression cassette consists essentially of a nucleic acid encoding a human Enterovirus CV-A16 P1 polypeptide, an EMCV IRES and an human Enterovirus EV-A71 3CD protease, which 3CD protease is under the translational control of the IRES, and which Enterovirus EV-A71 3CD protease processes the human Enterovirus CV-A16 P1 polypeptide into structural capsid polypeptides, which structural capsid polypeptides self-assemble into virus-like particles.

Reference may now be made to various embodiments of the invention as illustrated in the attached figures.

### Example 1. Construction of an Expression Cassette

All members of the genus Enterovirus, including EV-A71, polioviruses and CV-A16, have a single-stranded, positive sense RNA genome which has a single open reading frame encoding a polypeptide P1, consisting of the structural polypeptides VP0, VP1, VP2, VP3 and VP4 and several non-structural proteins including the viral proteases 3C and 3CD which are responsible for cleaving the polypeptide P1 into the individual structural capsid polypeptides, VP0, VP3 and VP1, wherein VP0 is eventually cleaved into VP4 and VP2.

Complete genome sequences of Enterovirus EV-A71 and CV-A16, as well as polioviruses are available in GenBank and are accessible at the National Center for Biotechnology Information (NCBI).

A recombinant DNA molecule encoding a P1 polypeptide may be constructed whereby open reading frames which encode Enterovirus structural polypeptides and proteases may be obtained by PCR amplification using suitably designed primers complementary to nucleic acid sequences of Enterovirus. Suitable primers may be designed according to standard techniques from publicly available nucleic acid sequences of Enterovirus such as those complete genome sequences which are available in GenBank and which are accessible at the National Center for Biotechnology Information (NCBI). Moreover, genetic sequences may be synthesized de novo according to technologies known in the art.

For example, GenBank accession numbers for the complete genome of Enterovirus EV-A71 include DQ341362, AB204852, AF302996 and AY465356; GenBank accession numbers for the complete genome of the human Enterovirus CV-A16 include KF924762.1; GenBank accession numbers for the complete genome of the human Enterovirus C poliovirus type I (PV1) genome include V01149 and V01150.

### Example 2. Construction of Expression Cassettes to Obtain CV-A16 VLPs

pSN01 has been used to generate a recombinant baculovirus harboring an expression cassette for the production of Enterovirus VLPs. The entry clone pSN01 originates from the work described in PCT Intemational Application No. PCT/IB2012/003114, see Example 1 and FIG. 1, and is the source of the baculovirus expression construct, SN07, described in PCT International Application No. PCT/IB2012/003114.

pSN01 harbors an expression cassette comprising a nucleic acid encoding an Enterovirus EV-A71 P1 polypeptide, an IRES, and a Enterovirus 3CD protease which derives from Enterovirus EV-A71.

pSN01 may be used to generate further expression cassettes comprising different P1 polypeptides. An example of such an expression cassette may be an expression cassette comprising a P1 polypeptide from CV-A16.

Extensive bioinformatics analyses were done to identify a consensus amino acid sequence for the CV-A16 P1 polypeptide. The CV-A16 P1 coding sequence was codon optimized for expression in insect cells and the P1 coding sequence was synthesized de novo by gene synthesis techniques known in the art.

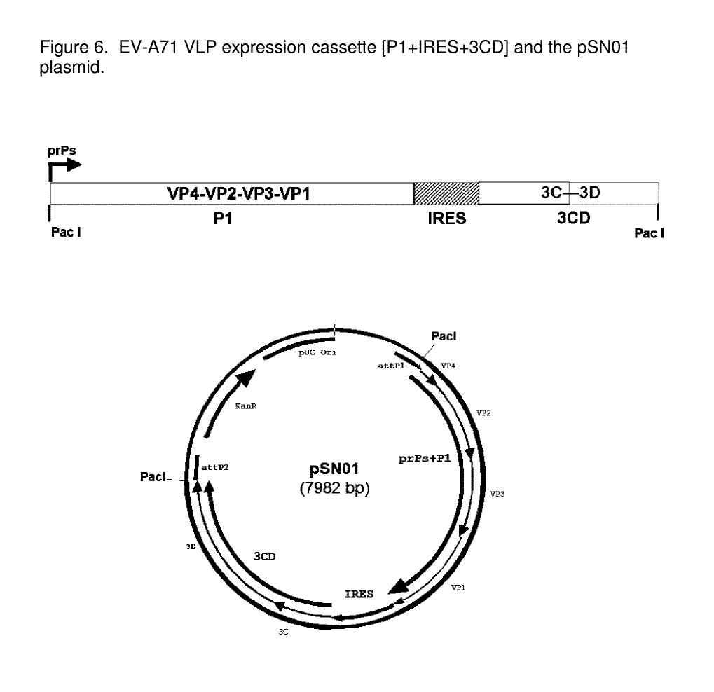

pSN01, depicted in FIG. 6, was used to generate an expression cassette where the P1 coding sequence of EV-A71 in pSN01 was replaced with the P1 coding sequence of CV-A16 by means known in the art.

For example, the codon optimized CV-A16 P1 gene was synthesized with a BgIII site upstream (5′) of the coding region, a partial IRES and BgII site downstream of the P1 stop codon. The synthesized DNA molecule was cloned into pUC57. The pUC57-CV-A16 P1-IRES(partial)-BgII plasmid was digested with BgII and BgIII and the BgII/BgIII DNA fragment containing CV-A16 P1-IRES(partial) was purified. pSN01 was digested with BgII and BgIII and the BgII/BgIII and the vector fragment of pSN01, wherein the EV-A71 P1-IRES(partial) has been removed, was purified and used as the vector for the CV-A16 P1 DNA fragment. The purified CV-A16 P1-IRES(partial) DNA fragment was cloned into the BgII/BgIII digested pSN01 vector giving rise to plasmid pSXT6.

pSXT6 comprises an expression cassette comprising a CV-A16 P1 polypeptide, an IRES, and a 3CD protease which derives from EV-A71.

A baculovirus expression construct comprising a CV-A16 P1 polypeptide, an IRES, and a CV-A16 3CD protease was obtained utilizing known CV-A16 genetic sequences encoding the P1 polypeptide and 3CD protease, wherein the nucleic acids encoding such P1 polypeptide and 3CD protease may be synthesized de novo according to techniques known in the art. The nucleic acids encoding the P1 polypeptide and the 3CD protease may be substituted for the P1 polypeptide and the 3CD protease in pSN01 according to methods known in the art and described herein.

pSXT9 comprises an expression cassette comprising a CV-A16 P1 polypeptide, an IRES, and a CV-A16 3CD protease.

pSN01, pSXT6 and pSXT9 were used to generate baculovirus expression constructs, the SN01 and SXT6 constructs harboring an expression cassette comprising an Enterovirus P1 polypeptide, an IRES, and a 3CD protease which derives from Enterovirus EV-A71. Methods used for generating the recombinant bacmids are as described in Invitrogen's GATEWAY® system instruction manual (Waltham, Mass.). pSN01, pSXT6 and pSXT9 were used to generate recombinant bacmids, which bacmids were sequence verified. Recombinant bacmids were purified using PureLink® HiPure Plasmid Miniprep (ThermoFisher Scientific, Waltham, Mass., USA), and then transfected into Sf9 cells following standard protocols, for example that protocol described in Invitrogen's Guide to Baculovirus Expression Vector Systems (BEVS) and Insect Cell Culture Techniques.

After 3 days, the supernatant was collected and designated passage 1 (p1) baculovirus stock. This is a small scale low titered baculovirus stock which was amplified by infecting Sf9 cells to generate a passage 2 (p2) baculovirus stock. The passage 2 baculovirus was used to infect Sf9 cells to generate passage 3 (p3) baculovirus stock, which was then used to evaluate expression of VLPs.

The recombinant baculovirus which was produced from the pSN01 is designated herein as SN07, and is described in PCT/IB2012/003114. The recombinant baculovirus which was produced by pSXT9 is designated as SXT9.

The recombinant baculovirus which was produced by bacSXT6 is designated as SXT6.

A schematic representation of the elements of the expression cassettes for constructs to provide CV-A16 VLPs is shown in FIG. 1. The nucleotide sequence of the expression cassette of the baculovirus SXT6 construct is provided as SEQ ID NO: 1.

### Example 3. Expression of CV-A16 Virus-Like Particles in Sf9 Cells Infected with Recombinant Baculovirus SXT6

Sf9 cells were infected with recombinant baculovirus SXT6 at a multiplicity of infection (MOI) of 0.01, and harvested on day 3 post-inoculation. The culture was subjected to centrifugation at 3100×g for 30 minutes (min) at 15° C. The pellet was washed once with PBS, resuspended in a hypotonic buffer (1.5 mM MgCl₂, 50 mM KCl, 20 mM HEPES) containing 0.1% Triton X100 and an EDTA-free protease inhibitor cocktail (Sigma) and then rocked for 30 min at room temperature. The cell lysate was then clarified by centrifugation at 6600×g for 20 min at 4° C.

Samples were separated by SDS-PAGE on a 12% gel and then electro-transferred to nitrocellulose membranes. The membranes were blocked in PBS containing 5% skim milk for 1 hour (hr) at room temperature (RT), and then probed overnight at room temperature with a mouse monoclonal antibody directed against Enterovirus A VP0 (MAb 979), a rabbit hyperimmune polyclonal antibody directed against Enterovirus VP1, or a rabbit hyperimmune polyclonal antibody directed against Enterovirus VP3. Bound antibodies were detected after incubation with anti-mouse IgG conjugated with horseradish peroxidase (HRP) for 1 hr, followed by 10 min incubation in TMB substrate at room temperature for colour development.

FIG. 4 shows that CV-A16 structural polypeptides VP0, VP1 and VP3 are present in the lysates of the Sf9 cells infected with the recombinant baculovirus SXT6 which carries the coding sequence of CV-A16 (P1) and the protease 3CD of EV-A71. The figure shows that the CV-A16 polypeptide is processed by the EV-A71 3CD protease to provide structural capsid polypeptides which assemble to form VLPs.

### Example 4. Antibodies are Generated Against CV-A16 VP1 and VPONP2 when VLPs Produced by Recombinant Baculovirus SXT6 are Used to Immunize Mice

Female Balb/c mice, 8-10 weeks old were immunized intraperitoneally with 200 pL of partially purified recombinant baculovirus generated SXT6 VLPs in the presence of Freund's Complete Adjuvant. Two weeks later, these mice were immunized with another 200 pL of partially purified recombinant baculovirus generated SXT6 VLPs in the presence of Freund's Incomplete Adjuvant. After two weeks, mice were euthanized and serum was harvested and stored at −80° C. for further analysis. Serum samples were incubated at 56° C. for 30 min before they were used in the immunoassays described next.

An indirect ELISA was performed using purified recombinant subunit capsid polypeptides, VP0, VP1 and VP3 from CV-A16. Wells were coated with the recombinant subunit capsid polypeptides overnight at 4° C. Wells were washed, blocked, and then incubated with various concentrations of serum from immunized mice for 1 hr at room temperature. The presence of CV-A16 specific antibodies was detected after incubation with HRP-conjugated anti-mouse-lgG for 1 hr at room temperature followed by addition of TMB substrate for 5 min at room temperature. The reaction was blocked by addition of 0.1N HCl. The absorbance was measured at a 450 nm wavelength.

Table 1 shows the titres of antisera from individual mice binding to the individual capsid proteins from CV-A16. The sera exhibited high titres ranging from 1:1000 to greater than 1:32,000 against VP0; ranging from 1:4000 to greater than 1:32,000 against VP1; and titres of at least 1:500 against CV-A16 VP3, with one of the mice having titres as high as 1:8,000. One mouse (Mouse 2) did not respond well, having titres to VP0 and VP1 below the limit of detection.

TABLE 1
Reciprocal titres of mouse antibodies
against capsid proteins of CV-A16.

| | CV-A16 VP0 | CV-A16 VP1 | CV-A16 VP3 |
|---|---|---|---|
| Mouse 1 | 32,000 | 4000 | 8,000 |
| Mouse 2 | <500 | <500 | 1,000 |
| Mouse 3 | >32,000 | >32,000 | 500 |
| Mouse 4 | 8,000 | 8,000 | 500 |
| Mouse 5 | 32,000 | 32,000 | 500 |
| Mouse 6 | 1,000 | <500 | 1,000 |
| Geometric mean titre | 7,127 | 4,326 | 1,000 |

This example shows that all major capsid proteins were expressed and able to elicit antibodies in mice.

### Example 5. VLPs Produced by Recombinant Baculovirus Generate Antibodies Directed Against CV-A16 Virus

Female Balb/c mice, 8-10 weeks old, were immunized intraperitoneally with 200 pL of partially purified SXT6 VLPs in the presence of Freund's Complete Adjuvant. Two weeks later, these mice immunized with another 200 pL of partially purified SXT6 VLPs in the presence of Freund's Incomplete Adjuvant. After two weeks, mice were euthanized and serum was harvested and stored at −80° C. for further analysis. Serum samples were incubated at 56° C. for 30 min before they were used in immunoassays.

An indirect ELISA was performed using infected cell lysates from CV-A16 infected rhabdomyosarcoma cells. Wells of the ELISA plate were coated with the infected cell lysates by incubating ovemight at 4° C. Wells were washed, blocked and then incubated for 1 hr at room temperature with serum from mice immunized with SXT6 VLPs diluted 1:100. The presence of CV-A16-specific antibodies was detected after incubation with HRP-conjugated anti-mouse-lgG for 1 hr at room temperature followed by addition of TMB substrate for 5 min at room temperature. The reaction was blocked by addition of 0.1N HCl. The absorbance was measured at a 450 nm wavelength.

FIG. 5 shows sera (diluted 1:100) from all mice immunized with recombinant baculovirus SXT6 VLPs and SXT9 VLPs bound to CV-A16-infected cell lysates, while the mice immunized with the control antigen, FGUS, did not bind significantly to the CV-A16-infected cell lysates. This shows that the SXT6 VLPs and SXT9 VLPs are able to elicit antibodies in mice which recognize antigens of native CV-A16 virus. SXT6 VLPs provide an unexpectedly improved immune response compared to that obtained by SXT9 VLPs.

### Example 6. Neutralizing Antibodies are Generated Against CV-A16 when VLPs Produced by Recombinant Baculovirus are Used to Immunize Mice

Plaque reduction neutralization test 50 (PRNT₅₀) was performed as follows. Serial dilutions of the mouse sera were incubated with 300 PFU/mL of EV-A71 or CV-A16 virus for 1 hr at 37° C. The serum-virus mixture was then added to Vero cell monolayers in a 24-well plate for 2 hr at 37° C. A carboxymethylcellulose overlay medium was added and the plate was incubated at 37° C. After 4 to 5 days post-inoculation, the monolayer was fixed and stained with naphthalene blue black and plaques were manually counted. The PRNT₅₀ titer is the lowest dilution of serum that results in >50% reduction in plaque number compared to the control wells inoculated with virus and no sera.

Table 2 shows the reciprocal PRNT₅₀ titre of the serum from each individual mouse immunized by the CV-A16 VLPs produced from the recombinant baculovirus SXT6. The sera from all mice tested were able to neutralize CV-A16 virus with titres ranging from 1:20 to 1:160, while sera from the mice immunized with a control baculovirus, FGUS, were unable to neutralize CV-A16 virus. The results demonstrate that the VLPs generated from recombinant baculovirus SXT6 contain functional CV-A16 neutralizing epitopes.

TABLE 2
Reciprocal PRNT₅₀ titres against CV-A16

| | CV-A16 Reciprocal Titre |
|---|---|
| SXT6 VLP Mouse 1 | 80 |
| SXT6 VLP Mouse 2 | Not done |
| SXT6 VLP Mouse 3 | 80 |
| SXT6 VLP Mouse 4 | 160 |
| SXT6 VLP Mouse 5 | 20 |
| SXT6 VLP Mouse 6 | 40 |
| Control mouse 7 | <20 (below limit of detection) |
| Control mouse 8 | <20 (below limit of detection) |

When the PRNT₅₀ titres were compared in sera obtained from control mice and mice immunized with SXT6 VLPs, the mice immunized with SXT6 VLPs produce neutralizing antibodies.

The present invention is not to be limited in scope by the specific embodiments described herein. Indeed, various modifications of the invention in addition to those described herein will become apparent to those skilled in the art from the foregoing description. Such modifications are intended to fall within the scope of the appended claims.

All patents, applications, publications, test methods, literature, and other materials cited herein are hereby incorporated by reference.

### REFERENCES

1. Ku Z, Liu Q, Ye X, Cai Y, Wana X, Shi J, Li D, Jin X, An W. Huana Z. A virus-like particle based bivalent vaccine confers dual protection against enterovirus 71 and coxsackievirus A16 infections in mice. Vaccine. 2014 Jul. 23; 32(34):4296-303. doi: 10.1016/j.vaccine.2014.06.025. Epub 2014 Jun. 17. PubMed PMID: 24950363.
2. Gong M. Zhu H. Zhou J. Yang C, Feng J. Huang X, Ji G, Xu H. Zhu P. Cryo-electron microscopy study of insect cell-expressed enterovirus 71 and coxsackievirus al 6 virus-like particles provides a structural basis for vaccine development. J Virol. 2014 June; 88(11):6444-52. doi: 10.1128/JVI.00200-14. Epub 2014 Mar. 26. PubMed PMID: 24672036; PubMed Central PMCID: PMC4093858.
3. Liu Q. Yan K, Fena Y, Huana X, Ku Z, Cai Y, Liu F. Shi J. Huana Z. A virus-like particle vaccine for coxsackievirus A16 potently elicits neutralizing antibodies that protect mice against lethal challenge. Vaccine. 30 (2012) 6642-6648.
4. Li Y, Zhu R, Qian Y. Dena J. The Characteristics of Blood Glucose and WBC Counts in Peripheral Blood of Cases of Hand Foot and Mouth Disease in China: A Systematic Review. PLoS ONE 7(1): e29003; published Jan. 3, 2012.
5. W U, et al., Vaccine 20, 895-904, 2002.
6. CHUNG, et al. World J Gastroenterol 12(6): 921-927, 2006.
7. CHUNG, et al. Vaccine 26:1855-1862, 2008.
8. CHUNG, et al. Vaccine 28:6951-6957, 2010.
9. MELOEN, et al., J. Gen. Virol. 45:761-763, 1979.
10. ZHU. et al., The Lancet 381:2024-2032, 2013.
11. ZHU., et al. New England Journal of Medicine 370(9):818-828, 2014.
12. L I, et al. New England Journal of Medicine 370(9):829-837, 2014.
13. ELLIS, Chapter 29 of Vaccines, PLOTKIN, et 1a. (eds) WB Saunders, Philadelphia, at page 571, 1998.
14. PLEVKA, et al. Proc. Natl. Acad. Sci. USA. 111(6):2134-9, 2014.
15. ROSSMANN, et al., Trends in Microbiology, Vol. 10(7) 324-331, 2002.

1-23. (canceled)

24. A Virus-Like Particle (VLP) which elicits a protective and/or neutralizing antibody response against infection by the Enterovirus from which the Enterovirus polypeptides of the VLP are derived.

25. The VLP of claim 24, wherein the Enterovirus is selected from Enterovirus A, Enterovirus B, Enterovirus C and Enterovirus D.

26. The VLP of claim 24, wherein the Enterovirus is CV-A16.

27. The VLP of claim 24, wherein the Enterovirus is selected from Poliovirus 1, Poliovirus 2 and Poliovirus 3.

28. A nucleic acid encoding an expression cassette comprising a promoter operably linked to a nucleic acid encoding an Enterovirus P1 polypeptide comprising structural polypeptides VP0, VP3 and VP1, which is operably linked to a nucleic acid encoding an Internal Ribosome Entry Site (IRES), and which is operably linked to a nucleic acid encoding an EV-A71 3CD protease.

29. The nucleic acid encoding the expression cassette of claim 28, wherein the Enterovirus P1 polypeptide is from an Enterovirus selected from Enterovirus A, Enterovirus B, Enterovirus C and Enterovirus D.

30. The nucleic acid encoding the expression cassette of claim 28, wherein the Enterovirus P1 polypeptide is from Enterovirus CV-A16.

31. The nucleic acid encoding the expression cassette of claim 28, wherein the Enterovirus P1 polypeptide is from an Enterovirus selected from Poliovirus 1, Poliovirus 2 and Poliovirus 3.

32. The nucleic acid encoding the expression cassette of claim 28, wherein the EV-A71 3C or 3CD protease in under translational control of the IRES.

33. The nucleic acid encoding the expression cassette of claim 28, wherein the nucleic acid sequence encoding the IRES has been genetically modified.

34. The nucleic acid encoding the expression cassette of claim 28, wherein the nucleic acid sequence encoding the 3CD has been genetically modified.

35. The nucleic acid encoding the expression cassette of claim 28, wherein the IRES is derived from Encephalomyocarditis virus (EMCV) or an Enterovirus.

36. A method of making an Enterovirus VLPs comprising the step of culturing a prokaryotic or eukaryotic host cell comprising a nucleic acid encoding the expression cassette of claim 28 for a period of time sufficient to produce Enterovirus P1 polypeptides and EV-A71 3CD proteases, and to form VLPs.

37. The method of claim 36, further comprising the step of recovering the VLPs from the host cell.

38. A composition comprising Enterovirus CV-A16 VLPs made by the method of claim 37, wherein the composition is essentially free of aggregates of Enterovirus CV-A16 VLPs.

39. A vaccine comprising the VLPs of claim 24.

40. A vaccine comprising VLPs obtained by the method of claim 37.

41. The VLPs of claim 24, for use in a vaccine for vaccinating a subject against infection by the Enterovirus, the use comprising administering to the subject the Enterovirus Virus-Like Particle in an amount effective to elicit an immune response and/or neutralizing antibody response directed against the Enterovirus from which the polypeptides of the VLP are derived when administered to the subject.

42. A method of providing an immune response and/or neutralizing antibody response against infection by an Enterovirus in a subject, the method comprising administering to the subject the Virus-Like Particles of claim 24 in an amount effective to provide such immune response and/or neutralizing antibody response against infection by an Enterovirus in the subject.
